(19) United States
(12) Patent Application Publication
(10) Pub. No.: US 2009/0035748 A1
(43) Pub. Date: Feb. 5, 2009

(54) BROMELAIN AS A CLINICAL SAMPLE PRE-TREATMENT, LYSIS AGENT AND NUCLEASE INHIBITOR

(76) Inventor: Tiffiny Marie LIZZI, Stewartstown, PA (US)

Correspondence Address: David W. Highet, VP & Chief IP Counsel;Becton, Dickinson and Company, (Birch Stewart Kolasch and Birch), 1 Becton Drive, MC 110, Franklin Lakes, NJ, 07417-1880, US

(21) Appl. No.: 11/832,508

(22) Filed: Aug. 1, 2007

(51) Int. Cl. C12Q 1/70; C12N 9/99; C12S 3/20; C12Q 1/68

(52) U.S. Cl. 435/5; 435/270; 435/184; 435/6

(57) ABSTRACT

This invention generally relates to the use of the proteolytic enzyme bromelain for treating samples for diagnostic assays. More specifically, the present invention relates to the use of bromelain in methods for pre-treating clinical samples primarily for the purpose of obtaining extractable and amplifiable DNA or RNA from prokaryotic or eukaryotic cells and/or viruses. The present methods also relate to using bromelain as a nuclease inhibitor. The use of bromelain as a nuclease inhibitor is useful in applications where the destructive nature of nucleases are detrimental to downstream applications. This invention also relates to the use of bromelain as a lysis agent for samples to be used, for example, in a molecular based diagnostic assay. The present methods include treating a sample which contains bacteria, viruses, host cells, fungi or parasites with bromelain, typically extracting nucleic acid from the sample, and subjecting the sample to a nucleic acid-based diagnostic assay.

### BACKGROUND

Bromelain is a cysteine protease derived from the stem of the pineapple plant (Ananas comosus). The activity of a cysteine protease is based upon nucleophilic attack of a cysteine thiol group from the protease on an amide bond of the substrate protein. This action results in hydrolysis of the amide bond to amine and carboxylic acid. Thus, bromelain breaks down proteins into smaller oligopeptides and/or amino acids.

Bromelain is a general name for a group of sulfhydryl proteolytic enzymes. Bromelain's primary component is a sulfhydryl proteolytic fraction. Crude bromelain is also known to contain a peroxidase, acid phosphatase, several protease inhibitors, and organically bound calcium. When the proteolytic fraction of bromelain is purified and extracted, the result is a potent proteolytic enzyme.

Diagnostic assays including obtaining or preserving nucleic acids from clinical samples that contain a large amount of protein could be greatly aided by pre-treatment with an agent that is capable of promoting the availability of the nucleic acids. In addition, current methods for the lysis of cells and recovery of nucleic acids frequently require the use of heat. The pre-treatment of samples with an agent that can aid in the cell lysis can reduce or eliminate the need for a heat lysis step and thus significantly decrease the time required to perform such assays.

### BRIEF DESCRIPTION OF THE DRAWINGS

FIG. 1: shows the results of a test of bromelain pre-treatment in a commercially available viral transport medium, M4RT® (Remel part number 12505) using Influenza B (INFB) linearized plasmid extracted on a Becton Dickinson Viper® instrument.

### DESCRIPTION OF THE INVENTION

Nucleic acid extraction. The present invention relates to methods for obtaining or preserving extractable and amplifiable nucleic acids from a sample containing prokaryotic or eukaryotic cells and/or viruses, which includes combining bromelain with a sample containing prokaryotic or eukaryotic cells and/or viruses and then extracting the nucleic acids. The present invention also preferably includes methods of treating non-tissue samples.

The term “combining” bromelain with a sample as used herein encompasses both adding bromelain to a sample, and adding a sample to bromelain. Combining also includes actively mixing, but combining also includes incubating without an active mixing step. The bromelain is inactivated by acid treatment in some embodiments of the invention. The term “extracting nucleic acid” as used herein is intended to encompass any of the methods generally known in the art for nucleic acid extraction from a sample.

While most proteolytic enzymes are not reactive unless used at temperatures above ambient, bromelain is effective at both elevated temperatures (˜45° C.) and room temperature. The term “room temperature” as used herein, is intended to be understood as is generally known in the art, and is usually between about 18° C. and 25° C. The use of bromelain thus results in improved efficiency in the overall extraction process. In addition, the effect of bromelain as a cysteine protease is important in the digestion of proteins, such as gelatin, that have varying degrees of tertiary structure.

Generally for obtaining or preserving extractable and amplifiable nucleic acids from a sample the amount of bromelain added per milliliter of sample is 0.01 to 100 U/mL. More preferably the amount is 0.05 to 50 U/ml. Most preferably the amount of bromelain added is 0.1 to 10 U/mL. Where one unit (U) will release 1.0 μmole of p-nitrophenol from Nα-Z-L-lysine p-nitrophenyl ester per min at pH 4.6 at 25° C. Alternatively, 1 Unit will hydrolyze 1.0 mg of amino nitrogen from gelatin in 20 minutes at pH 4.5 at 45° C.

The pH range is generally from about 3-10. More preferably the pH is between about 4-8, and most preferably the pH is between about 5-7. The temperature range is generally between about 20-80° C., more preferably between about 40-60° C. most preferably room temperature (about 18-25° C.).

Combining may or may not also include actively mixing with bromelain, and can involve only incubation. Mixing or incubation time is generally 5 minutes to 24 hours, preferably, 10 minutes to 1 hour. As noted, this invention does not require a mixing step. For example, approximately 20 minutes incubation time can be used with no mixing.

Combining may also be accomplished via an extended incubation. In this embodiment of the invention, the bromelain and sample are allowed to incubate in a vessel for an extended period of time which may be for 6, 12 hrs, or 24-48 hrs or more.

Nuclease inhibitor. The present invention also relates to using bromelain as a nuclease inhibitor. The use of bromelain as a nuclease inhibitor is particularly effective in applications where the destructive nature of nucleases would be detrimental to downstream applications.

The ability of bromelain to act as a protease allows it to be used to treat clinical samples, or specimen transport media which may contain proteins, including gelatin, in sufficient amounts to be inhibitory to downstream applications. It is known in the art that even trace amounts of gelatin are detrimental to magnetic particle extraction methods. By the addition of even very small amounts of bromelain to transport medium containing gelatin, efficiency of DNA and RNA extraction is significantly improved.

Accordingly, the present invention relates to a method for combining a biological sample which contain prokaryotic or eukaryotic cells and/or viruses to be used in a nucleic acid-based diagnostic assay, which includes combining the sample with bromelain, typically extracting nucleic acid from the sample, and subjecting the sample to a nucleic acid-based diagnostic assay.

As the tertiary structure of most RNases is stabilized by disulfide bonds, bromelain as a cysteine protease will disrupt these structures and render the RNases inactive. The ability of bromelain to remain active over both a large pH range, as well as a large temperature range, makes it an efficient nuclease inhibitor. See Khan et al., Effect of pH, temperature and alcohols on the stability of glycosylated and deglycosylated stem bromelain, J. Biosci. 28(6): 709-714 (2003), which is incorporated herein by reference in its entirety.

Generally for use as a nuclease inhibitor the amount of bromelain added per milliliter of sample is 0.01 to 001 U/mL. More preferably the amount is 0.05 to 50 U/ml. Most preferably the amount of bromelain added is 0.1 to 10 U/mL. One unit (U) will release 1.0 μmole of p-nitrophenol from Nα-Z-L-lysine p-nitrophenyl ester per min at pH 4.6 at 25° C. Alternatively, 1 Unit (U) will hydrolyze 1.0 mg of amino nitrogen from gelatin in 20 minutes at pH 4.5 at 45° C.

The pH range is generally from about 3-10. More preferably the pH is between about 4-8, and most preferably the pH is between about 5-7. The temperature range is generally between about 20-80° C., more preferably between about 40-60° C., most preferably room temperature (about 18-25° C.). Combining may or may not also include actively mixing with bromelain, and can include only incubating the sample. The mixing or incubating time is generally 5 minutes to 24 hours, more preferably, 10 minutes to 1 hour. As noted, this invention does not require an active mixing step. For example, 20 minutes incubation time can be used with no mixing.

Cell lysis. This invention also relates to the use of bromelain as an efficient lysis agent for clinical samples to be used, for example, in a molecular based diagnostic assay. Bromelain can be used to lyse host cells, fungi, and parasites, as well as bacteria and viruses. The present methods include treating a clinical sample which contains bacteria, viruses, host cells, fungi or parasites with bromelain, typically extracting nucleic acid from the sample, and subjecting the sample to a nucleic acid-based diagnostic assay.

Examples of molecular based diagnostic assays that can be used with this invention are the BD ProbeTec™ ET Chlamydia trachomatis (CT) and Neisseria gonorrhoeae (GC) Amplified DNA Assays made by Becton Dickinson & Company. Bromelain's ability to digest protein-based membranes and other cellular components allows for the availability of DNA or RNA for diagnostic purposes.

Bromelain aids in bacterial lysis for example for both CT and GC, for nucleic acid amplification, purposes. Bromelain reduces or eliminates the need for any heat lysis step in nucleic acid purification, and/or amplification, e.g. a 30 minute heat lysis step, and 15 minute cool down step which are typically required in the BD ProbeTec ET System, and thus dramatically increases amplification or assay efficiency.

The present invention provides methods in which assay sensitivity is greatly improved. The present invention also provides methods for the pre-treatment of certain clinical samples that either contain a large amount of protein, or that are collected in a transport medium that contains a large amount of protein. In one embodiment the present invention provides methods of using bromelain in plasma samples to digest proteins (to keep from congealing) at temperatures up to 75° C. Additional embodiments are also described herein. Unlike other proteolytic enzymes bromelain is inexpensive and readily available.

The methods of this invention may also include a step of removing the bromelain after sample pre-treatment. For example, a substance may be added to the sample that complexes with the bromelain and allows its removal by, for example, centrifugation. Alternatively, the bromelain may be inactivated by treatment at low pH, i.e. below a pH of about 3, or a high pH above 10.

Generally for use as a cell lysis agent the amount of bromelain added per milliliter of sample is 0.01 to 100 U/mL. More preferably the amount is 0.05 to 50 U/ml. Most preferably the amount of bromelain used is 0.1 to 10 U/mL. One unit (U) will release 1.0 μmole of p-nitrophenol from Nα-Z-L-lysine p-nitrophenyl ester per min at pH 4.6 at 25° C. Alternatively, 1 Unit (U) will hydrolyze 1.0 mg of amino nitrogen from gelatin in 20 minutes at pH 4.5 at 45° C.

The pH range is generally from about 3-10. More preferably the pH is between about 4-8, and most preferably the pH is between about 5-7. The temperature range is generally between about 20-80° C., more preferably between about 40-60° C., most preferably room temperature (about 18-25° C.). Combining can include actively mixing or incubating, and the mixing or incubating time is generally 5 minutes to 24 hours, more preferably, 10 minutes to 1 hour. As noted, this invention does not require a mixing step. For example, 20 minutes incubation time can be used with no mixing.

Bromelain. The bromelain used in this invention can be extracted from pineapple stems (CAS No: 37189-34-7). Generally, the bromelain that is used with this invention is obtained from any commercial source. Bromelain in the form of a powder, or lyophilized powder, or any other commercially available form can be used. For example, Sigma Chemical Co. provides bromelain extracted from pineapple stems as a lyophilized powder. (Product No. B4882). Fluka Chemical Co. also provides bromelain extracted from pineapple stems as a powder. (Product No. 16990). Generally the purity of the bromelain that is used with this invention may be of any commercially available grade.

Diagnostic assays. The molecular-based diagnostic assays that are contemplated by this invention generally include any molecular in vitro diagnostic assay. Subjecting a sample to a nucleic acid-based diagnostic assay as used herein is understood to mean performing a nucleic acid-based diagnostic assay on the sample in a manner generally known in the art. Preferably, the assays include or involve the extraction of DNA or RNA from a sample. In preferred embodiments, diagnostic assays for microbial pathogens including bacteria, fungi, viruses and parasitic organisms are contemplated in addition to assays for genetic markers of human or animal origin. Examples of diagnostic assays contemplated by this invention include the BD ProbeTec™ ET CT and GC Amplified DNA Assays for use with the BD Viper™ and BD ProbeTec™ ET Systems, both of which are manufactured by Becton Dickinson, & Company.

Other analytes for which the invention may be used include but are not limited to: respiratory viruses such as influenza A, B and respiratory syncytial virus; herpes viruses including herpes simplex viruses 1 and 2, human herpes viruses 6 and 8, Epstein Barr Virus, Varicella Zoster Virus, Human immunodeficiency virus (HIV), Hepatitis B Virus (HBV), and Cytomegalovirus; bacteria such as Mycoplasma spp., Legionella spp., Chlamydiaceae spp., Bordetella spp., Staphylococcus spp., Streptococcus spp., Mycobacterium spp., Gardnerella spp., Neisseria spp., Ureaplasma spp., and Enterococcus spp.; fungi including Candida spp. (in particular, Candida spp. involved in human disease) and Aspergillus spp.; and parasitic organisms including Trichomonas spp. and Treponema spp.

Samples. The samples that are treated by the present methods can generally include any biological samples that contain proteins, including gelatin, or nucleic acid of human or animal origin. The biological samples typically contain one or more prokaryotic cells, eukaryotic cells, or viruses and also include yeast. The term biological samples also includes, for example, samples from human cells for genotyping. Biological samples also include non-tissue samples that contain extractable and amplifiable DNA or RNA from bacteria, viruses, fungi, or parasites. Biological samples also include any clinical samples, or samples comprising specimen transport media which may contain proteins, including gelatin, in sufficient amounts to be inhibitory to downstream applications.

In certain preferred embodiments, the samples are non-tissue samples. Such non-tissue samples can contain extractable and amplifiable DNA or RNA from pathogens in transport media. Such samples contain, or are suspected of containing, bacterial, viral, fungal, or parasitic nucleic acid.

In another embodiment, this invention includes kits for carrying out the claimed methods. The kits include a vessel and bromelain contained in the vessel. The vessel may also optionally contain a stabilizer. The bromelain may be contained in the vessel in any convenient form including, but not limited to, powder, liquid, dried, incorporated in a dissolvable film, a tablet, or a dissolvable gel cap. The dried bromelain can be dried by any method known in the art, including but not limited to, air drying, vacuum dring, heat drying, and freeze drying. In a preferred embodiment the kit includes a test tube with dried bromelain and a stabilizer.

### EXAMPLE

A test was conducted of bromelain pre-treatment with M4RT medium using a linearized plasmid clone of an influenza nucleic acid sequence (INFB). The plasmid DNA was extracted from the sample using the BD Viper™ system was conducted. Strand displacement amplification of the extracted plasmid, conducted as known in the art, was used to evaluate extraction efficiency. The results shown below demonstrate that addition of bromelain to M4RT transport medium improves the recovery of amplifiable INFB target plasmid on the BD Viper SP® System.

Materials. Extraction tubes; INFB Priming microwells (PMW); Wash Buffer; Elution Buffer; Nuclease Free Water (Ambion); Acid; M4RT® medium (Remel); INFB Native Target Linearized Plasmid at 1×10⁶ copies/μl; 0.85% saline; Influenza Amplification Lot 085K1735.

Linearized INFB NT plasmid was diluted to working stock of 1×10³ copies/μl in a buffer solution comprised of Potassium Phosphate, pH7.6, Dimethyl sulfoxide (DMSO), and glycerol. This stock was boiled for 5 minutes and then cooled for 10 minutes at room temperature. A stock of bromelain powder was dissolved in 5 mL of M4RT medium to achieve a final concentration of 3 U/mL. This concentrated stock was then used to prepare two tubes of M4RT medium at a final concentration of 0.095 U/mL. The tubes of M4RT medium containing bromelain were incubated at 45° C. for 20 min, after which each tube was spiked with INFB Native Target (NT) linearized plasmid to achieve a final concentration of either 12.5 copies/μl or 25 copies/μl. Untreated M4RT medium was spiked with plasmid DNA at either 12.5 copies/μl or 25 copies/μl as a negative control. An untreated solution of 0.85% saline was spiked at 12.5 copies/μl plasmid DNA as a positive control.

One milliliter of each test solution was pipetted into appropriately labeled 4 mL tubes. Eight aliquots were tested from each condition. The tubes were then placed on the BD Viper instrument for extraction. In brief, the extraction process is comprised of a binding step, a wash step, and a nucleic acid elution step. First, acid is added to a tube containing the sample and magnetic particles. The pH is lowered and the nucleic acid is bound to the magnetic particles (ferric oxide). A wash step is performed on the magnetic particles and bound nucleic acids. An elution buffer is added to the sample tube, the pH is raised, and the nucleic acid is released from the magnetic particles. These released nucleic acids are now available for further amplification.

The eluate from the ferric oxide extraction was used to continue with the INFB strand displacement amplification (SDA) assay. Twenty microliters of Diluent A (comprised of Bicine, potassium hydroxide (KOH), DMSO, glycerol, magnesium acetate, and Proclin 300 was added to the INFB priming microwells (PMWs) containing amplification primers, fluorescent labeled detector probes, an enzyme necessary for the reverse transcriptase reaction, and other reagents necessary for amplification. Thirty microliters of each extraction eluate was added to the PMW, followed by 100 μl of Diluent B comprised of Bicine, KOH, DMSO, and Proclin 300. The contents of the PMW were then mixed by repeated aspiration and dispense with a multichannel pipette. The PMWs were incubated at room temperature for 20 minutes and then transferred to 72° C. heatblock. At the same time, amplification microwells (AMWs) were placed on a 54° C. heatblock and both microwell plates were incubated for a further 10 minutes. One-hundred microliters of the PMW reaction matrix was transferred to a corresponding AMW. The AMWs were sealed using an adhesive clear plastic sheet and incubated at 52° C. in a kinetic fluorescent reader (the BD ProbeTec ET System) for nucleic acid amplification and detection.

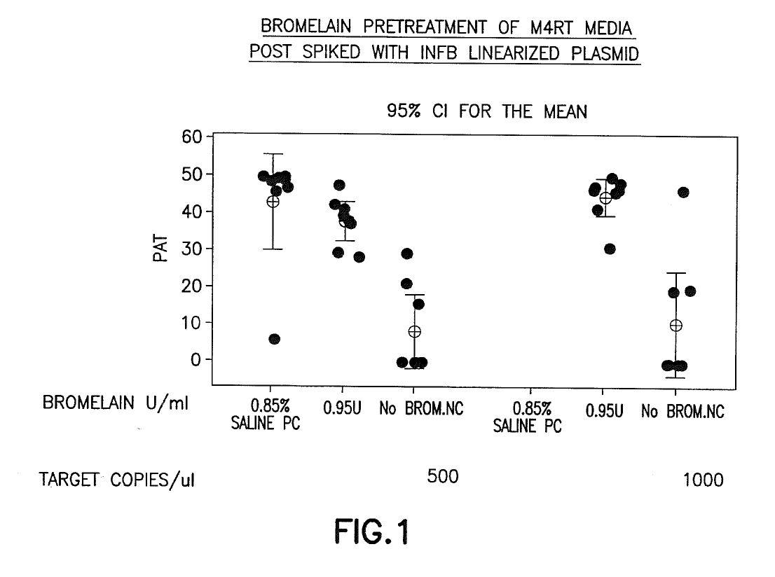

Results. Fluorescence was monitored over 60 passes of the instrument and results were expressed in terms of PAT scores (defined as 60-(number of passes required for relative fluorescent signal to pass a predetermined threshold)). PAT values equal to 0 were considered negative whereas PAT scores greater than 0 were considered positive. Positive PAT scores were achieved by pretreatment of respiratory transport medium with Bromelain prior to extraction. The untreated controls show that without bromelain, PAT scores were highly variable with several false-negative results. In contrast, all the spiked saline samples yielded positive results. FIG. 1 shows that both test conditions results fall within the 95% confidence interval of the positive control sample results.

TABLE 1
The results of SDA detection of INFB plasmid extracted from bromelain treated
M4RT medium using the BD Viper System.

| | Target Level | | | | |
|---|---|---|---|---|---|
| | 500 copies INFB | 1000 copies INFB | | | |
| | NT Plasmid/rxn | NT Plasmid/rxn | 500 copies/rxn | 1000 copies/rxn | 500 copies/rxn |
| | Media | | | | |
| | M4RT | M4RT | M4RT | | 0.85% Saline |
| | Brom. Conc. | | | | |
| | | | No Brom. Neg | No Brom. Neg | No Brom. |
| | 0.095 U/mL | 0.095 U/mL | Cntrl. | Cntrl. | Pos. Cntrl |
| PAT results | 37.2 | 46.7 | 0 | 0 | 49.7 |
| | 47.4 | 47.6 | 15.9 | 20.2 | 49.3 |
| | 39.3 | 46.1 | 0 | 0 | 48.5 |
| | 37.9 | 31.4 | 0 | 46.9 | 46.8 |
| | 42.3 | 41.5 | 0 | 19.8 | 45.7 |
| | 41.3 | 46.8 | 29.5 | 0 | 48.5 |
| | 29.6 | 48.7 | 0 | 0 | 49.6 |
| | 28.3 | 50.1 | 21.3 | 0 | 5.8 |
| Avg | 37.9 | 44.9 | 8.3 | 10.9 | 43.0 |
| SD | 6.4 | 6.0 | 12.1 | 17.1 | 15.1 |
| CV | 16.8 | 13.3 | 144.8 | 157.8 | 35.1 |

We claim:

1. A method for obtaining or preserving nucleic acids from a biological sample comprising:
combining bromelain with a biological sample; and
extracting nucleic acids from the sample.

2. The method of claim 1, wherein the sample contains a bacterial cell or a virus.

3. The method of claim 1, wherein the amount of bromelain used is between 0.01 U/mL and 100 U/mL.

4. The method of claim 1, wherein the amount of bromelain used is between 0.1 U/mL and 10 U/mL.

5. The method of claim 1, wherein the bromelain is combined with the sample at room temperature.

6. The method of claim 1, wherein the bromelain is combined with the sample at a temperature of about 40-80° C.

7. A method for inhibiting the activity of a nuclease in a biological sample comprising:
combining a sample which contains one or more nucleases with bromelain.

8. The method of claim 7, wherein the amount of bromelain used is between 0.01 U/mL and 100 U/mL.

9. The method of claim 7, wherein the amount of bromelain used is between 0.1 U/mL and 10 U/mL.

10. The method of claim 7, wherein combining the sample with bromelain is performed at room temperature.

11. A method for performing cell lysis on a biological sample to be used in a nucleic acid-based diagnostic assay comprising:
combining the sample with bromelain;
extracting nucleic acid from the sample; and
subjecting the sample to a nucleic acid-based diagnostic assay.

12. The method of claim 11, wherein the sample comprises Chlamydia trachomatis, Neisseria gonorrhoeae, influenza A, B, respiratory syncytial virus; herpes virus, herpes simplex viruses 1 and 2, human herpes viruses 6 and 8, Epstein Barr Virus, Varicella Zoster Virus, Human immunodeficiency virus (HIV), Hepatitis B Virus (HBV), Cytomegalovirus, Mycoplasma spp., Legionella spp., Chlamydiaceae spp., Bordetella spp., Staphylococcus spp., Streptococcus spp., Mycobacterium spp., Gardnerella spp., Neisseria spp., Ureaplasma spp., Enterococcus spp.; Candida spp., Candida spp. involved in human disease, Aspergillus spp., Trichomonas spp. or Treponema spp.

13. The method of claim 11, wherein combining the sample with bromelain is performed at room temperature.

14. The method of claim 11, wherein the nucleic acid-based diagnostic assay is a nucleic acid amplification-based assay.

15. The method of claim 11, wherein both combining the sample with bromelain and the nucleic acid amplification-based assay are conducted at room temperature.

16. The method of claim 11, wherein combining the sample with bromelain comprises incubating with no mixing.

17. A kit comprising:
a vessel; and
bromelain contained in the vessel.

18. The kit of claim 18 wherein the tube further contains a stabilizer.
